(19) United States
(12) Patent Application Publication
(10) Pub. No.: US 2009/0018442 A1
(43) Pub. Date: Jan. 15, 2009

(54) Diagnostic medical ultrasound system having clock synchronized power supply

(76) Inventors: Gregg Miller, Boulder, CO (US); Todd D. Willsie, Seattle, WA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/827,804

(22) Filed: Jul. 13, 2007

(51) Int. Cl. A61B 8/00

(52) U.S. Cl. 600/437

(57) ABSTRACT

A system and method is disclosed for minimizing image artifacts, or the effect thereof, of electrical noise/interference caused by switch-mode power supplies of a diagnostic medical imaging system, such as a diagnostic medical ultrasound system. In one embodiment, one or more switch mode power supplies capable of being synchronized to an external clock signal are utilized to power the ultrasound system. The clock signal to the power supplies is provided by the system, generated, or otherwise derived from, the clock signal used by the imaging components, and phase-synchronized to the transmit and receive events of the imaging system. In this way, noise generated by the power supplies is synchronized with the imaging processes and, at least, will appear as a substantially constant artifact in the diagnostic images, allowing it to be readily ignored by a user or automatically removed therefrom.

### BACKGROUND

Diagnostic medical ultrasound systems are routinely used in medical applications for the purpose of imaging various body tissues and organs and for other diagnostic and therapeutic purposes. These systems allow medical professionals to view the internal conditions of a patient thereby enabling them to render a better diagnosis. In one example of a diagnostic medical ultrasound system, a piezoelectric transducer acquires image data by transmitting a series of ultrasonic pulses into a patient and receiving the echoes therefrom. These echoes are converted/manipulated into an image and displayed on a monitor or stored for later use.

As technologies for implementing diagnostic medical ultrasounds systems become more integrated and physical system dimensions are reduced, the introduction of electrical noise/interference, such as that caused by the system's power supplies, into the imaging processes becomes problematic. Artifacts created by such noise may appear in the diagnostic images and may mislead a user or otherwise impede or interfere with a diagnosis. With smaller systems, physical limitations, such as size or weight limitations or the proximity of components to each other, may preclude the use, or reduce the effectiveness, of traditional noise suppression techniques, such as electro-magnetic or radio frequency shielding, or providing power supply conditioning.

Accordingly, there is a need for improved electro-magnetic noise/interference management to minimize image artifacts.

### BRIEF DESCRIPTION OF THE DRAWINGS

FIG. 1 depicts a representation of one embodiment of an ultrasound system having a clock synchronized power supply.

FIG. 2 depicts a representation of one embodiment of the power supply synchronization logic of the system of FIG. 1.

FIG. 3 depicts timing diagrams for exemplary power supply clock signals generated by the logic of FIG. 2.

FIG. 4 depicts a flow chart showing operation of the power supply synchronization logic of FIG. 2.

### DETAILED DESCRIPTION OF THE PRESENTLY PREFERRED EMBODIMENTS

The disclosed embodiments relate to minimizing image artifacts, or the effect thereof, of electrical noise/interference caused by switch-mode power supplies of a diagnostic medical imaging system, such as a diagnostic medical ultrasound system. Such noise/interference may be rendered substantially identifiable within the diagnostic images such that it may be ignored by the user or removed, automatically or manually from the images, such as by inverse signal cancellation or other image processing techniques. In one embodiment, one or more switch mode power supplies capable of being synchronized to an external clock signal are utilized to power the ultrasound system. The clock signal to the power supplies is provided by the system, generated or otherwise derived from the clock signal used by the imaging components, and phase-synchronized, e.g. phase-locked, to the transmit and receive events of the imaging system. In this way, noise generated by the power supplies is synchronized with the imaging processes and, at least, will appear as a substantially constant artifact in the diagnostic images, allowing it to be readily ignored by a user or automatically removed therefrom. Further, variances over time, such as image to image, in the phase of the power supply switching is eliminated, thereby reducing the appearance of image artifacts due to such variances.

A switch or switched mode power supply is an electronic power supply unit (PSU) that incorporates a switching regulator—an internal control circuit that switches power transistors (such as MOSFETs) rapidly on and off in order to stabilize the output voltage or current. The current in a SMPS is switched on and off sharply, and contains high frequency spectral components. This high-frequency current can generate undesirable electromagnetic interference. In particular, such interference or noise may occur or be more apparent when the power supply is switching out of phase with the timing of the rest of the system.

One exemplary switch mode power supply is the TPS6205X800-mA Synchronous Step-Down Converter, manufactured by Texas Instruments, Inc., located in Dallas, Tex. The TPS6205x devices are a family of high-efficiency synchronous step-down dc/dc converters ideally suited for systems powered from a 1-cell or 2-cell Li-Ion battery or from a 3-cell to 5-cell NiCd, NiMH, or alkaline battery. The TPS62050 is a synchronous PWM converter with integrated N-channel and P-channel power MOSFET switches. Synchronous rectification increases efficiency and reduces external component count. The exemplary device can be synchronized to an external clock signal in the range of 600 kHz to 1.2 MHz. The device automatically detects the rising edge of the first clock and synchronizes to the external clock.

In typical ultrasound systems, the switched mode power supplies are asynchronous to the clock signals used by the rest of the system, e.g. they are generated internal to the power supplies themselves, and accordingly these clock signals are not synchronized with each other or with the rest of the system. Therefore, the noise generated by the power supplies, which will generally be generated synchronous with the power supply clock signals, may occur at random phases as compared with the transmit/receive event cycles which are synchronized with the system clock signals. As such, if not suppressed via other means, such as electro-magnetic shielding, the image artifacts created by this noise/interference may appear to move or otherwise appear to the user in some varied fashion, e.g. out of phase, and be difficult to distinguish from the imaged anatomical features, even if the power supply and system clock signals are operating at the same frequency. In one example, during Doppler color imaging, such varying noise may be mis-interpreted as blood flow. It will be appreciated that the disclosed embodiments are applicable to other types of power supplies as well which may be used in lieu of or in addition to switch mode power supplies, such as a fly-back topology based power supply. The disclosed embodiments may be applied to any type of power supply which operates based on an internal or external clock signal. In embodiments utilizing both clocked and non-clocked power supplies, the disclosed methodology may only be applied to the clocked power supplies.

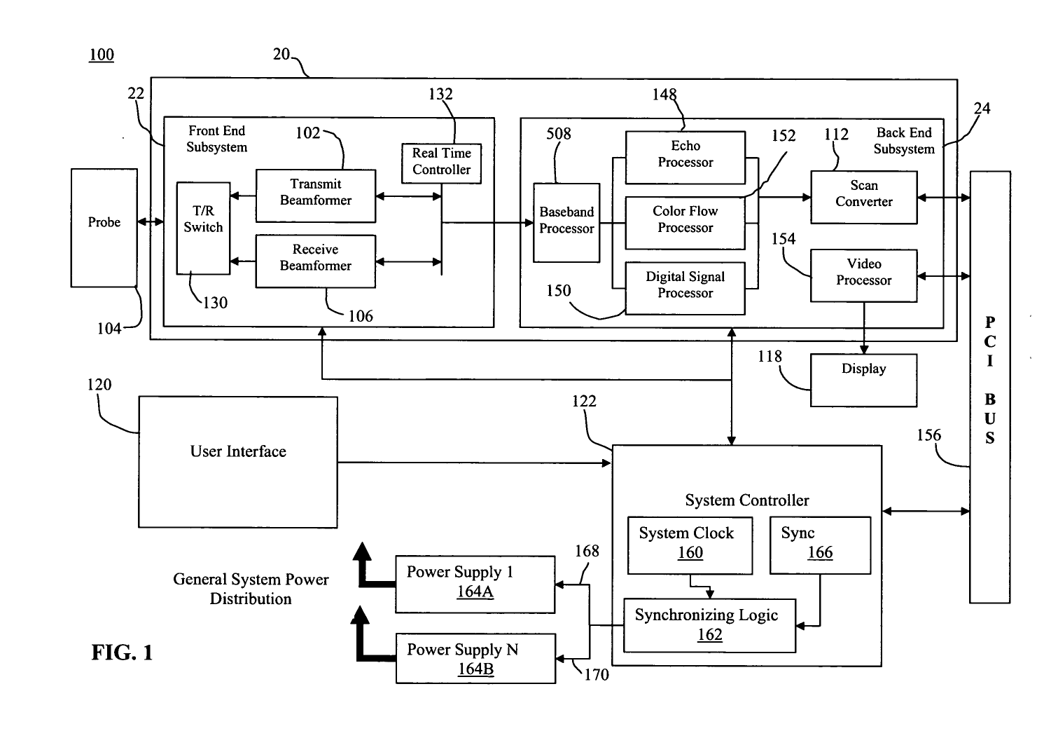

FIG. 1 shows a block diagram of an architecture of one embodiment of a diagnostic medical ultrasound system 100. It will be appreciated that the disclosed embodiments may be applicable to other imaging systems such as computed radiography, magnetic resonance, angioscopy, color flow Doppler, cystoscopy, diaphanography, echocardiography, fluoresosin angiography, laparoscopy, magnetic resonance angiography, positron emission tomography, single-photon emission computed tomography, x-ray angiography, computed tomography, nuclear medicine, biomagnetic imaging, culposcopy, duplex Doppler, digital microscopy, endoscopy, fundoscopy, laser surface scan, magnetic resonance spectroscopy, radiographic imaging, thermography, radio fluroscopy, or any combination thereof.

In one embodiment, the system 100 is a cart based imaging system. In another embodiment, the system 100 is a portable system, such as a briefcase-sized system or laptop computer based system. Other embodiments include handheld ultrasound systems. For example, one or more housings are provided where the entire system is small and light enough to be carried in one or both hands and/or worn by a user. The system may have any weight, such as a handheld system weighing less than 6 pounds or weighing less than 2 pounds. In another example, a transducer is in one housing to be held by a person, and the imaging components and display are in another housing to be held by a person. Coaxial cables connect the two housings. A single housing for an entire handheld system may be provided. One or more of the housing may contain a portable power source which provides source of electrical power for the system 100. Alternatively, or in addition thereto, a connection may be provide for an external power source such as a line/wall based power source, external battery pack, or the like.

The depicted architecture corresponds to the architecture of the Acuson P10™ Portable Ultrasound Platform manufactured by Siemens Medical Solutions USA, Inc., located in Iselin, N.J. It will be appreciated that one or more of the described components may be implemented in integrated within or divided among one or more hardware components, software components or a combination thereof. The ultrasound system 100 includes an ultrasonic imaging probe or transducer 104, acquisition hardware 20, a front end acquisition hardware subsystem 22, a back end acquisition hardware subsystem 24, a user interface 120, a system controller 122 and a display 118. In addition, one or more power supplies 164A, 164B are provided to provide the requisite electrical power to the various components described herein. The power supplies 164A, 164B may supply power from a battery or capacitive based power source, a line/wall based power source, or other internal or external power source or combination thereof. While the distribution of the electrical power from the power supplies 164A, 164B is not shown, it will be apparent to one of skill in the art, depending upon the implementation, as to the various methods available for routing electrical power. In one embodiment, the back end subsystem 24 comprises a baseband processor 108, an echo processor 148, a color flow processor 228, a digital signal processor 206, a scan converter 112 and a video processor 220. In one embodiment, the exemplary front end acquisition hardware 22 includes a transmit beamformer 102, a receive beamformer 106, a transmit/receive switch 130, and a real time controller 132. As will be discussed below, the front end acquisition hardware 22 may alternatively include local or remote optical or magnetic data storage devices such as a computer memory, hard disk, CD, DVD or video tape recorder coupled with the ultrasound system 100 via a wired or wireless device or network interface. Herein, the phrase “coupled with” is defined to mean directly connected to or indirectly connected through one or more intermediate components. Such intermediate components may include both hardware and software based components.

The front end acquisition hardware 22 is coupled with the transducer 104. The front-end acquisition hardware 22 causes the transducer 104 to generate acoustic energy in a subject and receives the electrical signals generated by the transducer 104 in response to the received echoes. In one embodiment, the front end acquisition hardware 22 is configurable to acquire information corresponding to a plurality of two-dimensional representations or image planes of a subject for three-dimensional reconstruction. Other configurations, such as those for acquiring data with a two dimensional, 1.5 dimensional or single element transducer array, may be used. To generate each of the plurality of two-dimensional representations of the subject during an imaging session, the acquisition hardware 20 is configured to transmit, receive and process during a plurality of transmit and receive events. Each transmit event corresponds to firing acoustic energy along one or more ultrasound scan lines into a portion of the subject. As a result of the succession of transmit events occurring during use of the system 100, information is received continuously throughout this process, e.g. receive events.

The transmit beamformer 102 is coupled with the transducer 104 and is of a construction known in the art, such as a digital or analog based beamformer capable of generating signals at different frequencies. The transmit beamformer 102 generates one or more excitation signals which causes the transducer 104 to emit one or more ultrasonic pulses. Each excitation signal has an associated center frequency. As used herein, the center frequency represents the frequency in a band of frequencies approximately corresponding to the center of the amplitude distribution. Preferably, the center frequency of the excitation signals is within the 1 to 15 MHz range and accounts for the frequency response of the transducer 104. The excitation signals have non-zero bandwidth.

It will be appreciated that alternative methods of generating and controlling ultrasonic energy as well as receiving and interpreting echoes received therefrom for the purpose of diagnostic imaging, now or later developed, may also be used with the disclosed embodiments in addition to or in substitution of current beamforming technologies. Such technologies include technologies which use transmitters and/or receivers which eliminate the need to transmit ultrasonic energy into the subject along focused beam lines, thereby eliminating the need for a transmit beamformer, and may permit beam forming to be performed by post processing the received echoes. Such post-processing may be performed by a receive beamformer or by digital or analog signal processing techniques performed on the received echo data. For example, please refer to U.S. patent application Ser. No. 09/518,972, entitled “METHOD AND APPARATUS FOR FORMING MEDICAL ULTRASOUND IMAGES”, now U.S. Pat. No. 6,309,356 and U.S. patent application Ser. No. 09/839,890, entitled “METHOD AND APPARATUS FOR FORMING MEDICAL ULTRASOUND IMAGES”, the disclosures of which are herein incorporated by reference.

Control signals are provided to the transmit beamformer 102 and the receive beamformer 106 by the real time controller 132. The transducer 104, as controlled by the transmit beamformer 102, is caused to fire one or more acoustic lines in each transmit event, and the receive beamformer 106 is caused to generate in-phase and quadrature (I and Q) information along one or more scan lines during receive event. Alternatively, real value signals may be generated. A complete frame of information corresponding to a two-dimensional representation (a plurality of scan lines) is preferably acquired before information for the next frame is acquired. The real time controller 132 is also used to manage the data flow created by the receive beamformer as it collects image information, making the stream of data available to the back end subsystem 22.

Upon the firing of one or more ultrasound scan lines into the subject, some of the acoustical energy is reflected back to the transducer 104, referred to as the receive event. This reflected acoustical energy is detected by the transducer 104 and converted into electrical signals which are passed to the receive beamformer 106. In addition to receiving signals at the fundamental frequency (i.e., the same frequency as that transmitted), the non-linear characteristics of tissue or optional contrast agents also produce responses at harmonic frequencies. Harmonic frequencies are frequencies associated with non-linear propagation or scattering of transmit signals. As used herein, harmonic includes subharmonics and fractional harmonics as well as second, third, fourth, and other higher harmonics. Fundamental frequencies are frequencies corresponding to linear propagation and scattering of the transmit signals of the first harmonic. Non-linear propagation or scattering corresponds to shifting energy associated with a frequency or frequencies to another frequency or frequencies. The harmonic frequency band may overlap the fundamental frequency band.

The baseband processor 108 is coupled with the receive beamformer 106 and receives the converted electrical signals representative of the reflected acoustical energy. The baseband processor 108 passes information associated with a desired frequency band, such as the fundamental band or a harmonic frequency band. In one embodiment, the baseband processor 108 may be included as part of the receive beamformer 106. Furthermore, the baseband processor 108 demodulates the summed signals to baseband. The demodulation frequency is selected in response to the fundamental center frequency or another frequency, such as a second harmonic center frequency. For example, the transmitted ultrasonic waveforms are transmitted at a 2 MHz center frequency. The summed signals are then demodulated by shifting by either the fundamental 2 MHz or the second harmonic 4 MHz center frequencies to baseband (the demodulation frequency). Other center frequencies may be used. Signals associated with frequencies other than near baseband are removed by low pass filtering. As an alternative or in addition to demodulation, the baseband processor 108 provides band pass filtering. The signals are demodulated to an intermediate frequency (IF) (e.g. 2 MHz) or not demodulated and a band pass filter is used. Thus, signals associated with frequencies other than a range of frequencies centered around the desired frequency or an intermediate frequency (IF) are filtered from the summed signals. The demodulated or filtered signal is passed to the additional processors 148, 228 and 206 as either the complex I and Q signal or other types of signals, such as real value signals. It should be noted that band pass “filtering”, as well as other types of data filtering known in the art, should not be confused with the filter elements of the pipes and filters framework disclosed herein. As known in the art, “filtering” data involves allowing data with certain characteristics to pass while blocking data without those characteristics. On the other hand, while the filter elements discussed below may perform functions similar to those provided by the band pass processor 108, the filter elements, as used by the architecture described herein, are more general processing stages that manipulate, transform or enrich streaming data.

By selectively filtering which frequencies are received and processed, the backend subsystem 22 produces images with varying characteristics. In tissue harmonic imaging, no additional contrast agent is added to the target, and only the nonlinear characteristics of the tissue are relied on to create the ultrasonic image. Medical ultrasound imaging is typically conducted in a discrete imaging session for a given subject at a given time. For example, an imaging session can be limited to an ultrasound patient examination of a specific tissue of interest over a period of ¼ to 1 hour, though other durations are possible.

Tissue harmonic images provide a particularly high spatial resolution and often possess improved contrast resolution characteristics. In particular, there is often less clutter in the near field. Additionally, because the transmit beam is generated using the fundamental frequency, the transmit beam profile is less distorted by a specific level of tissue-related phase aberration than a profile of a transmit beam formed using signals transmitted directly at the second harmonic.

The harmonic imaging technique described above can be used for both tissue and contrast agent harmonic imaging. In contrast agent harmonic imaging, any one of a number of well known nonlinear ultrasound contrast agents, such as micro-spheres or the Optison™ agent by Nycomed-Amersham of Norway, are added to the target or subject in order to enhance the non-linear response of the tissue or fluid. The contrast agents radiate ultrasonic energy at harmonics of an insonifying energy at fundamental frequencies.

The echo 148, color flow 152 and digital signal 150 processors are coupled with the baseband processor 108 and receive the filtered signals from the transducer 104/receive beamformer 106. The digital signal processor 150 comprises one or more processors for generating two-dimensional Doppler or B-mode information. For example, a B-mode image, a color Doppler velocity image (CDV), a color Doppler energy image (CDE), a Doppler Tissue image (DTI), a Color Doppler Variance image, or combinations thereof may be selected by a user. The digital signal processor 150 detects the appropriate information for the selected image. In one embodiment, the digital signal processor 150 is adapted for Doppler processing and a B-mode processing. As known in the art, the Doppler processing estimates velocity, variance of velocity and energy from the I and Q signals. As known in the art, the B-mode processing generates information representing the intensity of the echo signal associated with the I and Q signals. The echo processor 148 performs baseband and amplitude mode signal processing of RF and IQ data in a known manner. The color flow processor 152 adds color to the acquired information, as known in the art.

The information generated by the echo 148, color flow 152 and digital signal 150 processors is provided to the scan converter 112. Alternatively, the scan converter 112 includes detection processes as known in the art and described in U.S. Pat. No. 5,793,701 entitled “METHOD AND APPARATUS FOR COHERENT IMAGE FORMATION”, assigned to the assignee of the present invention, the disclosure of which is herein incorporated by reference. The scan converter 112 is of a construction known in the art for arranging the output of the signal processors 148, 228 and 206 into two-dimensional representations or frames of image data. The scan converter 112 converts acoustic ultrasound line data, typically in a polar coordinate system, into data which may be plotted on a Cartesian grid. Using volume averaging or other similar algorithms on the returned echo data, the slice information is merged into a single 2D plane. This permits display of the ultrasound image on a two-dimensional output device such as a display monitor 118. The scan converter 112 outputs formatted video image data frames, using a format such as the DICOM Medical industry image standard format or a TIFF format. Thus, the plurality of two-dimensional representations is generated. Each of the representations corresponds to a receive center frequency, such as a second harmonic center frequency, a type of imaging, such as B-mode, and positional information. The harmonic based representations may have better resolution and less clutter than fundamental images. By suppressing the harmonic content of the excitation signal, the benefits of harmonic imaging of tissue may be increased. In any event, the scan converter 112 provides its output to the PCI bus 210. In one embodiment, the PCI bus 210 is a standard peripheral component interconnect board, as known.

The user interface 120 is coupled with the system controller 122 and includes one or more input devices which the clinician/sonographer/physician uses to interface with the ultrasound system 100. The user interface 120 includes input devices such as a keyboard, mouse, trackball, touch screen or other input devices or combinations thereof as are known in the art. Further the user interface 120 may also include graphic user interface (“GUI”) elements coupled with the input devices and with the display 118 for both input and output functions. In addition to controlling the ultrasound functions of the ultrasound system 100, the user interface 120 may afford the user the opportunity to modify graphical representations, imaging planes and displays produced by the ultrasound system 100. Finally, the user interface 120 may allow the user to coordinate multiple ultrasound probes 104.

The system controller 122 is coupled with the front end subsystem 22, the backend subsystem 22, the PCI bus 210 and the user interface 120 and controls and coordinates the functions of the ultrasound subsystems. The term “system controller” broadly refers to the appropriate hardware and/or software components of the ultrasound system 100 that can be used to implement the embodiments described herein. It should be understood that any appropriate hardware (analog or digital) or software can be used and that the embodiments described herein can be implemented exclusively with hardware. Further, the system controller 122 can be separate from or combined with (in whole or in part) other processors of the ultrasound system 100 (including attendant processors), which are not shown in FIG. 1 for simplicity.

The various elements of the ultrasound system including the front end subsystem 22, backend subsystem 24 and user interface 120 are controlled in real time by the system controller 122. The system controller 122 controls the operation of the components of the system 100. A user, via the user interface 120, can adjust imaging parameters such as, but not limited to, image depth, image width, and frame rate. The controller 122 interprets the set-up information entered by the user and configures the components of the system 100 accordingly.

The video processor 154 acts as an interface between the system controller 122 and the display 118. In various embodiments, the video processor 154 can be configured to work with a variety of display types, such as cathode ray tubes or liquid crystal displays (LCD). The video processor 154 can also be configured to output information to a printer, memory, storage device, such as a computer storage device or a video recorder, computer network or other means for communicating data representative of an ultrasonic echo known in the art. The display monitor 118 is connected to the display controller 116 and is a standard display monitor, such as a color LCD, as known in the art. In alternate embodiments, the display 118 can be replaced with a printer, memory, storage device, or any other output device known in the art.

As discussed above, the ultrasound system 100 includes one or more switch-mode power supplies 164A, 164B which supply the requisite electrical power to the various components of the system 100. Multiple power supplies 164A, 164B may be provided to supply the different requisite electrical voltages needed by different components. In one embodiment, four power supplies 164A, 164B are provided. It will be appreciated that the number of power supplies and the voltages supplied therefrom is implementation dependent and that any number of power supplies supplying any variation of electrical power are contemplated. The power supplies 164A, 164B are responsive to external clock signals and, as described above, synchronize their switching activity according thereto. It will be appreciated that other types of power supplies 164A, 164B may be used in lieu of or in addition to switch mode power supplies, such as a fly-back topology based power supply. The disclosed embodiments may be applied to any type of power supply which operates based on an internal or external clock signal. In embodiments utilizing both clocked and non-clocked power supplies, the disclosed methodology may only be applied to the clocked power supplies.

The system controller 122 further includes a system clock 160 and power supply synchronizing logic 162. The system clock 160 generates the clock control signals generally for the clocked components of the ultrasound system 100 and may include multiple clock signal generators or oscillators generating clock signals of differing frequencies. In one embodiment, the system clock 160 provides a clock signal to the synchronizing logic 162 which is the same clock signal to which transmit and receive events are synchronized. The system controller 122 further generates or derives a sync signal 166 which is also coupled with the synchronizing logic 162 and which is indicative of the initiation of a transmit and/or receive event, or multiples thereof. The sync signal 166 may be internally generated by the system controller 122 or derived from an external source. In one embodiment, the sync signal 166 is derived from the horizontal sync signal generated by the video processor 154 and/or scan converter 112 and which may be used to generate images on the display 118 synchronous to the transmit and/or receive events. In systems 100 having digital displays, such as an LCD, as opposed to a cathode-ray-tube based display, a horizontal sync signal is typically superfluous but may still be provided. It will be appreciated that any signal synchronous with transmit and/or receive event(s), or multiples or combinations thereof, may be used directly or in derivative form, as the sync signal 166 for the purposes of synchronizing the power supplies 164A, 164B as described, such as a start of frame indicator. Further, the signal may be indicative of the occurrence of multiple receive events for a single transmit event, multiple transmit events for a single, e.g. composite, receive event, or combination thereof.

The synchronizing logic 162 is further coupled with each of the power supplies 164A, 164B to provide the clock signals thereto. Each power supply 164A, 164B may require a specific clock frequency, as a multiple of the system clock 160, and accordingly the synchronizing logic 162 may provide individual clock signals 168, 170 to each power supply 164A, 164B, or groups thereof. In one embodiment having four power supplies 164A, 164B, the requisite clock signals include a 606 Khz signal used by three of the four power supplies 164A, 164B and a 178 Khz, used by the fourth power supply 164A, 164B.

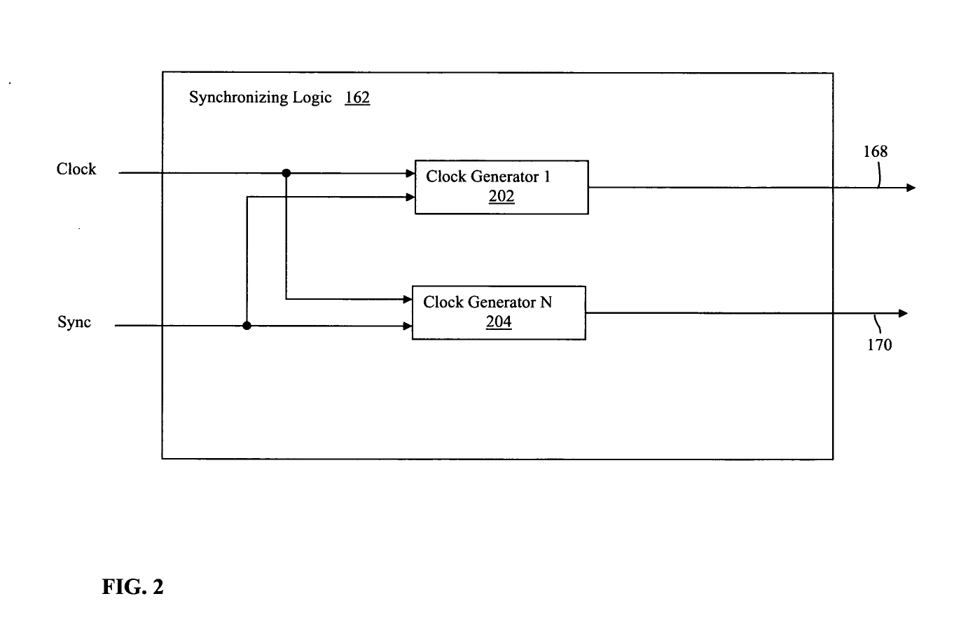

FIG. 2 shows a block diagram of one embodiment of the synchronizing logic 162 of FIG. 1. The synchronizing logic 162 include one or more clock generating circuits 202, 204 each coupled with the system clock 160 input and sync signal 166 input and outputting a clock signal 168, 170 to one or more of the power supplies 164A, 164B. Each clock generating circuit 202, 204 may include one or more state machines, counters/clock-dividers, clock-multipliers, or combinations thereof, which are capable of generating the unique frequencies, in phase with the system clock 160, as required by each power supply 164A, 164B. Each clock generator 202, 204 is responsive to the sync signal 166 so as reset to a start condition that corresponds to the beginning of the transmit/receive sequence for each ultrasound beam, thereby resetting the phase of the clock signals 168, 170. The clock generators 202, 204 may be implemented in hardware, software or a combination thereof, such as by combinational logic implemented on an integrated circuit.

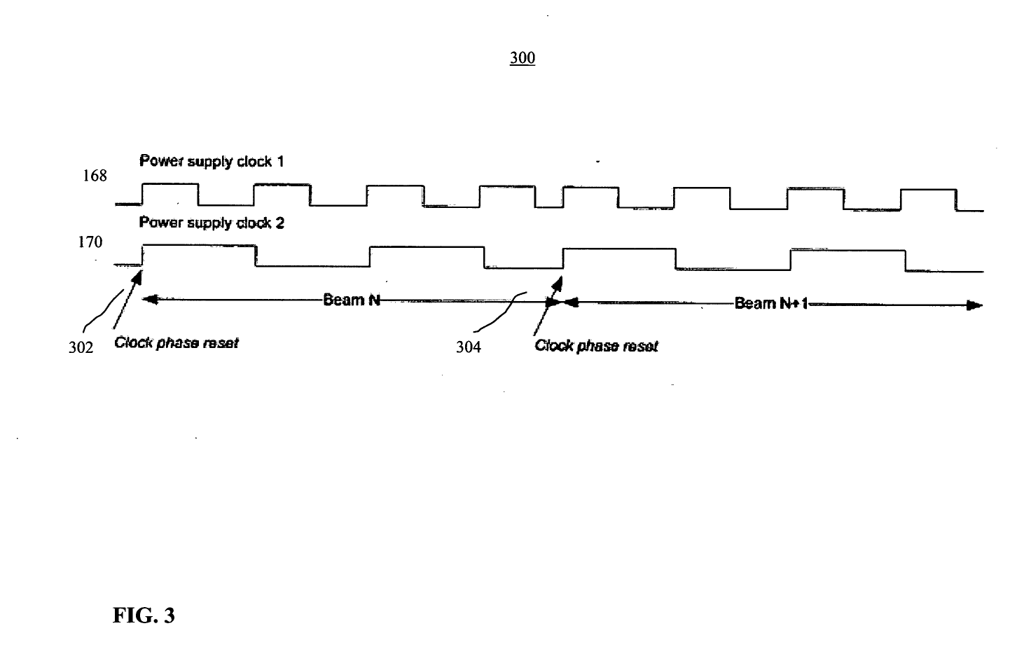

FIG. 3 shows a timing diagram 300 of the clock signals 168, 170 demonstrating how the phase of the signal is reset to synchronize with the initiation of the transmit/receive events 302, 304.

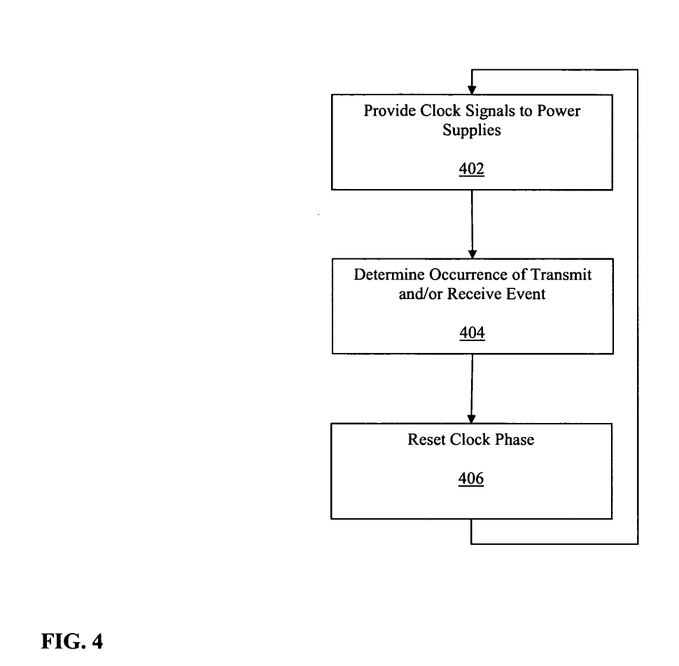

FIG. 4 shows a flow chart depicting exemplary operation of the synchronizing logic 162. Generally, the synchronizing logic 162 generates various multiples of the system clock 160 and provides these clock signals 168, 170 to the power supplies 164A, 164B (Block 402). Responsive to these signals 168, 170, the power supplies 164A, 164B, control their switching operations as discussed above. When it is determined that a transmit and/or receive event has been initiated, such as by a signal, e.g. the horizontal sync signal (block 404), the synchronizing logic 162 resets the phase of the signals 168, 170 so as to synchronize the clock signals 168, 170 to the occurrence of the transmit/receive event. Thereby, the switching operations of the power supplies 164A, 164B also synchronize to the occurrence of the transmit and/or receive events.

Thereby, the disclosed embodiments at least cause image artifacts due to power supply noise to appear in phase, e.g. phase-locked, with the system operation and, thereby, at least appear as a constant in the generate images. This permits the artifacts to be readily identified and ignored or removed, such as by processing the image. In color mode, when looking at the phase shift of the returning signal, by finding the difference between and ensemble of returning echoes from the same place in the body, any change in noise “phase” from a clock that is not synchronized, will result in noise in the image. Accordingly, synchronizing the phase of the power supply operation render image artifacts due to such noise easy to identify and further, noise may be minimized by eliminating variances in the power supply clock phase over time, such as image to image. In pulse inversion harmonic mode, the system 100 transmits a signal and its inverse into the subject and subtracts the resulting received echoes. If the noise from the power supplies is phase-locked, as described above, using the disclosed synchronous power supply clocking, then the noise will cancel out in this mode. For normal B-mode imaging, the disclosed synchronization technique may allow for removal, automatic or manual, of the resulting image artifacts, as they will appear in the same place image to image.

For portable ultrasound systems, where the physical limitations of portability constrain the physical dimensions of the system, the ability to utilize traditional methods to mitigate electrical noise/interference, e.g. electro-magnetic/RF shielding and/or power conditioning, is limited. The disclosed embodiments take advantage of the component proximity in compact systems which may make the system clock signals for readily accessible and which may further allows the power supplies to receive the system clock signal over a relatively short signal path, minimizing signal trace/routing complexity and/or the necessity or length of additional wiring, reducing clock skew, etc.

It is therefore intended that the foregoing detailed description be regarded as illustrative rather than limiting, and that it be understood that it is the following claims, including all equivalents, that are intended to define the spirit and scope of this invention.

We claim:

1. A diagnostic medical imaging system comprising:
a power supply operatively responsive to a power supply clock signal;
an event indicator operative to generate an event signal indicative of an occurrence of one of a transmit event, a receive event or a combination thereof; and
a power supply synchronizer coupled with the event indicator and the power supply and operative to reset the power supply clock signal based on the event signal.

2. The diagnostic medical imaging system of claim 1, wherein the power supply synchronizer is further operative to synchronize a phase of the power supply clock signal to the occurrence of the one of the transmit event, the receive event or combination thereof.

3. The diagnostic medical imaging system of claim 2, wherein the one of the transmit event, the receive event or combination thereof is synchronized with a system clock signal characterized by a first frequency and wherein the power supply clock signal is characterized by a second frequency different from the first frequency.

4. The diagnostic medical imaging system of claim 1, wherein event signal is further indicative of the initiation of the one of the transmit event, the receive event or combination thereof.

5. The diagnostic medical imaging system of claim 1, wherein the power supply comprises a switch mode power supply operative to switch responsively to an external clock signal.

6. The diagnostic medical imaging system of claim 1, wherein the diagnostic medical imaging system comprises a diagnostic medical ultrasound system.

7. The diagnostic medical imaging system of claim 1, wherein the transmit event comprises transmission of energy into a portion of a subject.

8. The diagnostic medical imaging system of claim 1, wherein the receive event comprises detection of an emission of energy from a portion of a subject.

9. The diagnostic medical imaging system of claim 1, wherein the event signal comprises an indicator of the display of an image generated as a function of one of the transmit event, the receive event or combination thereof.

10. The diagnostic medical imaging system of claim 9, wherein the display of each generated image is controlled by a display signal, the event signal being based thereon.

11. The diagnostic medical imaging system of claim 10, wherein the display signal comprises a horizontal sync signal that is indicative of the horizontal refresh of the display of the generated image.

12. The diagnostic medical imaging system of claim 1 wherein the system is characterized by a weight of less than or equal to 6 pounds.

13. The diagnostic medical imaging system of claim 1 wherein the system is characterized by a weight of less than or equal to 2 pounds.

14. A method of minimizing artifacts caused by noise in images generated by a diagnostic medical imaging system, the method comprising:
providing a power supply clock signal to a power supply of a diagnostic medical imaging system, the power supply operating responsively thereto;
determining the occurrence of one of a transmit event, receive event or a combination thereof; and
resetting the power supply clock signal based on the occurrence.

15. The method of claim 14, wherein the resetting further comprises synchronizing a phase of the power supply clock signal to the occurrence of the one of the transmit event, the receive event or combination thereof.

16. The method of claim 15, wherein the one of the transmit event, the receive event or combination thereof is synchronized with a system clock signal characterized by a first frequency and wherein the power supply clock signal is characterized by a second frequency different from the first frequency.

17. The method of claim 14, wherein the determining of the occurrence of one of the transmit event, the receive event or combination thereof further comprises determining when the one of the transmit event, the receive event or combination thereof is initiated.

18. The method of claim 14, wherein the power supply comprises a switch mode power supply capable of switching responsively to an external clock signal.

19. The method of claim 14, wherein the diagnostic medical imaging system comprises a diagnostic medical ultrasound system.

20. The method of claim 14, wherein the transmit event comprises transmission of energy into a portion of a subject.

21. The method of claim 14, wherein the receive event comprises detection of an emission of energy from a portion of a subject.

22. The method of claim 14, wherein each of the generated images is displayed as a function of one of the transmit event, the receive event or combination thereof, wherein the determining of the occurrence of the one of the transmit event, the receive event or combination thereof is based on the display of each generated image.

23. The method of claim 22, wherein the display of each generated image is controlled by a display signal, wherein the determining of the occurrence of the one of the transmit event, the receive event or combination thereof is based on the display signal.

24. The method of claim 23, wherein the display signal comprises a horizontal sync signal that is indicative of the horizontal refresh of the display of the generated image.

25. A diagnostic medical imaging system comprising:
means for providing a power supply clock signal to a power supply of a diagnostic medical imaging system, the power supply operating responsively thereto;
means for determining the occurrence of one of a transmit event, receive event or a combination thereof; and
means for resetting the power supply clock signal based on the occurrence.

26. A method of minimizing artifacts caused by noise in images generated by a diagnostic medical ultrasound system, the method comprising:
determining the start of one of the transmission of ultrasonic energy into a portion of a subject or the start of the reception of echoes from the portion of the subject due to the prior transmission of ultrasonic energy into the portion of the subject; and
synchronizing a phase of a clock signal to a switched mode power supply of the diagnostic medical ultrasound system based on the determining, the operation of the switched mode power supply synchronizing thereto.
